(19) United States
(12) Patent Application Publication
(10) Pub. No.: US 2009/0165723 A1
(43) Pub. Date: Jul. 2, 2009

(54) METHODS OF PRODUCING AVIAN EGGS AND BIRDS OF SPECIFIED GERM-FREE STATUS

(75) Inventor: Leonard Moran, County Mayo (IE)

Correspondence Address: K&L Gates LLP, STATE STREET FINANCIAL CENTER, One Lincoln Street, BOSTON, MA, 02111-2950, US

(73) Assignee: OVAGEN INTERNATIONAL LIMITED, County Mayo (IE)

(21) Appl. No.: 12/279,600

(22) PCT Filed: Feb. 22, 2007
(86) PCT No.: PCT/EP2007/051737

§ 371 Date: Oct. 22, 2008

(30) Foreign Application Priority Data
Feb. 23, 2006 (EP) 2006/060243

(51) Int. Cl. A01K 45/00; A61K 31/47; C12N 5/06; A01K 67/027

(52) U.S. Cl. 119/6.8; 514/312; 435/349; 800/8

(57) ABSTRACT

The invention provides an improved method of rearing a bird of specified germ-free status. Specifically, the present invention is directed to a method for increasing the hatchability and viability of surgically derived eggs of germ-free status when obtained from a parent bird. The method comprises housing a bird as a parent bird, determining the position of a premature egg in the reproductive tract and stage of development of the premature egg in terms of shell formation and calcification, surgically removing an egg in its shell from the parent bird prior to transfer of the egg to the cloaca in the parent bird, incubating the egg and hatching the egg to produce a laying bird. The invention also relates to the production of avian eggs of specified germ-free status and uses thereof.

### FIELD OF THE INVENTION

The present invention relates to an improved method of rearing a bird of specified germ-free status. Specifically, the present invention is directed to a method for increasing the hatchability and viability of eggs removed in a sterile manner from the abdomen of a parent bird and the egg is of germ-free status. It further relates to the production of avian eggs of specified germ-free status.

In this specification, the terms “contamination free” and “germ-free” are used interchangeably. These terms are used very broadly and relate to many pathogens and infections that can be carried by birds, particularly poultry such as chickens, turkeys and other avian species, which are used widely to produce flocks of birds for breeding to produce fertile eggs for commercial production and to produce eggs and meat for human consumption. Further, such eggs and birds are used in the manufacture of a wide range of biological substances including vaccines, antibodies, monoclonal antibodies, fibroblasts and proteins, both for therapeutic and prophylactic use in people and animals. They are further used extensively for diagnostic tests and the production of transgenic eggs and birds. Many of these uses require eggs and/or the birds produced from them to be free of either all or specified contaminants such as infections, including a variety of species of parasite, bacteria, mycoplasma, viruses, retroviruses, prions, DNA and RNA fragments. Sometimes, the viruses can be small viruses including picorna and parvo viruses. Some of the bacteria from which eggs are often contaminated include Clostridia and Enterobacteria. There are many non-pathogenic organisms that should be controlled. Similarly, many of the micro-organisms which include parasites, aerobic and anaerobic bacteria, commensal species and species associated with the gut and cloaca, are undesirable. Similarly, mycoplasma, viruses including retroviruses, prions, fungi, yeast and moulds are also undesirable.

Therefore, the term “specified contamination free” or “germ-free status” could include some or all of these and is much broader than just free of specified pathogens. For example, conventional specific pathogen free (SPF) are not specified free from some viruses and indeed can be contaminated with bacteria and viruses. Thus, for certain uses, these may be sufficient. The use to which the eggs and the birds are to be put will determine the contaminants that the egg or bird must be free of. Conventional germ-free and some SPF eggs are derived by treating fresh naturally laid eggs with chemicals, including disinfectants and antibiotics, and placing them in isolators. Such naturally laid eggs are taken from selected parent stock birds. While these methods have been relatively successful in the production of SPF eggs, they have not been truly successful in producing what are contaminant free eggs. However, the chemicals are not able to eliminate contamination from, for example, bacteria entering the pores of the eggshell immediately before and/or after laying and before disinfection. Contamination of eggs, whether SPF, germ-free or gnotobiotic, results in loss of compliance with specifications and, in many instances, loss of commercial value and utility.

### BACKGROUND TO THE INVENTION

European Patent no. 0 295 964 describes an in-vitro avian embryo culture technique describes in some detail the embryonic development of eggs. This specification is directed to the incubation of an embryo in a closed container after the embryo has been removed from its shell. Indeed, in this specification, the container used is preferably part of an egg shell which has been chosen from the same species as is being cultured or, in the terms of the present invention, from a similar hen. This invention is directed towards the genetic engineering of poultry but also to the investigation of fundamental mechanisms of avian development. Similarly, European Patent no. 0 511 431 discloses an in-vitro culture method for a fertilised ovum of a hen in which an embryo which has just been fertilised is taken from an upper portion of the magnum of the oviduct of a hen within an hour or so after oviposition and then subsequently cultured. However, both of these specifications merely disclose the artificial culturing of eggs and do not deal with the purpose of the present invention.

European patent application no. 01650109, is directed to a method of rearing a bird of specified contamination free status. The general method disclosed in this application comprises housing a bird as a parent bird, sterile removal of an egg from the parent bird prior to transfer of the egg to the cloaca in the parent bird, incubating the egg and hatching the egg to produce a laying bird. The application also relates to the production of avian eggs of specified contamination free status.

However, there is always a need for improvement of such methods. It is desirable to increase the hatchability rates and decrease the mortality of the live chicks. It is commercially very desirable to obtain both consistent results and results that include high hatchability (ideally approaching commercial ranges of >85% but certainly consistently >50%). This is of particular and vital importance when working with small, rare and highly valuable populations, such as for example transgenic birds.

The present invention, in contrast to the previous application, provides improved methods to obtain consistently high hatchability of germ-free eggs. Thus, the present invention is directed to a refinement and improvement of the process as claimed in European patent application no. 01650109.

### Statement of the Invention

The present invention is directed towards an improved method of rearing a bird of specified germ free status. Specifically, this invention relates to the application of specific techniques which may be used to ensure that the premature egg is removed in its shell prior to entry to the cloaca and remains sterile in accordance with the invention.

According to a more general embodiment of the present invention, there is provided a method for determining when removal of the premature egg in its shell from the parent bird should take place.

According to a first aspect of the invention, there is provided a method for increasing the hatchability and viability of surgically derived eggs of germ-free status when obtained from a parent bird wherein the method is carried out in a sterile environment prior to removal of a premature egg from a parent bird and which includes the following steps:
a) Housing a bird as a parent bird;
b) Determining the timing of when to remove the premature egg from the parent bird by:
   i. Determining the position of the premature egg in the reproductive tract of the parent bird prior to transfer of the premature egg to the cloaca of the parent bird; and
   ii. Establishing when the requisite level of egg-shell formation and calcification has taken place by determining the stage of development of the premature egg in terms of shell formation and calcification.

According to a second aspect of the invention, there is provided a method of rearing a bird of germ-free status comprising, in a sterile environment
a) housing a bird as a parent bird;
b) Determining the timing of when to remove the premature egg in its shell from the parent bird by:
   i. Determining the position of the premature egg in the reproductive tract of the parent bird prior to transfer of the premature egg to the cloaca of the parent bird; and
   ii. Establishing when the requisite level of egg-shell formation and calcification has taken place by determining the stage of development of the premature egg in terms of shell formation and calcification.
c) removing the premature egg in its shell from the parent bird when the requisite level of egg-shell formation and calcification has taken place and prior to transfer of the premature egg to the cloaca in the parent bird; and
d) incubating the premature egg in its shell and hatching the premature egg to produce a laying bird.

According to a third aspect of the invention, there is provided a method for the removal of micro-organisms which may infect the premature egg whilst it is in the reproductive tract, including the ovary, prior to the egg entering the cloaca.

According to a fourth aspect of the invention, eggs produced in accordance with this invention are used in the production of biological substances for therapeutic and prophylactic purposes.

According to a fifth aspect of the invention, the method of the invention is used to provide sterile birds for replenishment of bird stocks.

### DETAILED DESCRIPTION OF THE INVENTION

The cloaca is the principle area of contamination within a bird. The cloaca is a chamber linked to both the digestive and reproductive systems of the bird, therefore an egg and faeces may be present in the cloaca at the same time. In the main, the egg, prior to entering the cloaca is free of contamination. However, as an eggshell is porous for a short time, external contamination when it enters the cloaca is a major problem. Thus, this invention has recognised that specific methods are required to remove the premature egg from the parent bird whilst maintaining its sterility and germ-free status.

Specifically, the present invention has recognized the need to improve and refine the method for determining when removal of the premature egg in its shell from the parent bird should take place. Accordingly, the present invention provides a method for increasing the hatchability and viability of derived eggs of germ-free status when obtained from a parent bird. Any increase in hatchability and/or viability of the egg of germ-free status will be of significant commercial importance, even if the percentage increases are relatively modest.

The present invention has also recognized that the removal of micro-organisms, bacteria, mycoplasma, viruses, retroviruses, prions, parasites, which are able to infect the egg whilst in the reproductive tract and prior to the egg entering the cloaca is important in the generation of a germ-free egg.

Specifically, this invention recognizes the need to determine accurately the position and stage of development of the premature egg in the reproductive tract before completion of its development in the parent bird and prior to removal from the parent bird. This ensures that the premature egg is removed from the parent bird at the correct time to ensure a viable egg and laying bird are produced. If the premature egg is removed too early, it could have a soft, uncalcified shell which would require more specialised culture conditions and could also not result in a viable egg. Removal of the premature egg from the reproductive tract prior to entry to the cloaca prevents contamination of the egg and the bird produced therefrom. This invention provides specific methods to establish when to remove the egg from the parent bird. and ensures that the premature egg is removed from the parent bird at the correct time in terms of shell formation and calcification to result in a viable egg and laying bird.

Generally, prior to removal of the egg, the laying pattern of the parent bird is recorded over time to produce an estimated transfer time of the egg to the distal reproductive tract and cloaca. At this stage, the egg and its shell are preferably fully formed. This provides a guide so that sterile removal of the egg from the abdomen may be carried out as close to but prior to the estimated transfer time as possible.

Ideally, the eggshell should be calcified to an advanced stage which may be quantified using diagnostic methods such as radiography or even by gentle digital pressure, palpation, and observation of the distortion of the shell. Partially calcified shells and in particular shells that have low levels of calcification will usually be soft and fragile and hence have low hatchability levels. The present invention recognizes that premature eggs should not be removed at this stage if optimum hatchability and viability of the resultant egg and birds is desired.

According to one embodiment of the invention, the method for defining the stage of development, particularly in relation to shell deposition and position in the reproductive tract prior to removal of the egg from the parent bird may comprise observation, by either continuous observation or time-lapse video recording of the normal frequency (e.g. daily) and time of laying within either the normal or a modified cycle of laying, as applicable to the species and individual bird. Observation of when and how long each egg resides in different sections of the reproductive tract including the uterus relative to time of ovulation and of egg laying. Correlation of these different sets of observations enables prediction of not only, for example, when the next egg will reside in the uterus but also its stage of development, for example the degree of egg-shell formation and calcification.

This method can in certain of the larger species (e.g. chicken) be either refined by physical location of the egg in the abdomen. This approach is particularly useful for routine use after establishing the necessary correlation between palpated observations, stage of egg development and position in the reproductive tract

Confirmation of the position of the egg in the reproductive tract, and presence of a shell by physical methods including abdominal and pelvic palpation, visualisation techniques, such as ultrasound, X-ray or MRI, and/or direct observation by general anaesthesia and surgery or post mortem examination may also be used in combination with the observation techniques or on their own.

Visualisation techniques normally require general anaesthesia of the bird using, for example halothane and oxygen. Veterinary equipment used for visualisation of cats and dogs is often suitable for birds after systematic adjustments to exposure settings. Ultrasound is only possible in birds with few or no abdominal feathers.

Palpation should usually be performed with the bird held in approximately the normal standing position. Palpation of the abdomen is made between an operators' thumb and fingers. It should be performed gently to avoid any damage to either the bird or the eggs within the abdomen.

Eggs should not be removed from the bird before shell calcification if broadly conventional incubation and hatching conditions are to be used subsequently. More premature eggs, such as those with soft, uncalcified shells or earlier require specialised culture conditions and/or recipient eggshells. If uncertainty exists for a particular bird and egg, removal of the egg should be delayed until the egg observed has been laid. A subsequent egg may then be removed.

These techniques involve a subjective assessment by the person carrying out the procedure. Palpation determines whether the egg is soft and uncalcified or not. This technique may be combined with techniques to determine the position of the egg within the abdomen. The egg should be located before the pelvic outlet. Preferably, the egg should be substantially halfway between the caudal aspect of the sternum and the tuber ischii. This distance varies from bird to bird. Preferably, palpation is used in combination with X-ray, MRI or ultrasound visualisation techniques.

According to one embodiment of the invention, the method comprises a combination of observation and/or physical methods. Ideally, said physical methods include abdominal and pelvic palpation, ultrasound, X-ray and/or MRI scanning. Ideally, the observation methods include the steps of monitoring and recording the laying pattern of the parent bird over time to produce an estimated transfer time. The selection of the most appropriate method will depend on the manually dexterity and skill of the operator in palpation as well as other factors such as the size of the bird. Additional refinement may be obtained by careful observation of the individual bird to determine the time during the day that a bird usually lays an egg; a target is to remove the egg close to and slightly before anticipated egg laying.

According to another embodiment of the invention, there is provided a method for preventing infection of a premature egg by micro-organisms which may infect the egg whilst in the reproductive tract (including the ovary) but prior to the egg entering the cloaca. This type of infection may be via a trans-ovarian route. The micro-organisms can be either prevented from gaining entry to the developing egg or be removed from the unlaid egg by administration, to either the parent bird and/or the egg, of antimicrobials as appropriate to the target micro-organisms. This embodiment provides a method for the removal of contamination from the unlaid premature egg by the selection of antimicrobials known to be active against the target micro-organism in ova and their administration to the parent bird or the unlaid egg.

Selection of the correct antimicrobial and its dose, regimen and route of administration are based on in ova concentrations and time obtained in ova specific to the particular stage of development of the egg. These dosage regimens and routes may differ from those more typically used in routine treatment of common diseases.

The method generally comprises identification of the target micro-organisms by use of standard microbial identification laboratory techniques and then selection of appropriate antimicrobials to kill the micro-organisms.

Selection of the antimicrobials, dosage regimen and route of administration may be determined by standard in vitro sensitivity results as often used routinely in clinical microbiological laboratories operating to national and international standards such as CLS. For optimal results, in ova determination of antimicrobial concentrations and time relative to time of dosing and the specific development stage of the egg should be used. In ova concentrations of individual antimicrobials require pharmacokinetic data for egg concentrations determined in serial samples of eggs at different stages of development. This can be determined by administering a known dose of antimicrobial to laying hens which are then euthanased at appropriate serial time points (depending on the administration of the antimicrobial for example, 0.5, 1, 2, 4, 8, 12, 20 and 24 hours after administration) and appropriate samples of eggs collected post mortem. Antimicrobial concentrations in eggs should be determined using conventional methods adapted and specifically validated for use with egg material.

Fluroquinolone, cephalosporin and macrolide antimicrobials may be used to decrease or eliminate bacteria and mycoplamsas, depending on antimicrobial sensitivity and safety in the bird species. Fluroquinolone antimicrobials such enrofloxacin should be administered to achieve concentration-dependent bacterial or mycoplamsal killing, and use dosage regimens of at least those recommended for routine therapeutic use. For example, enrofloxacin at 10-30 mg/kg/day administered in water during a 2-5 h period. Cephalosporin and macrolide antimicrobials should use dosage regimens to ensure maximum exposure based on time-dependent bacterial killing. Other classes of antimicrobials may also be used.

Such micro-organisms can be either prevented from gaining entry to the developing egg or be removed from the unlaid egg by administration, to either the parent bird and/or the egg, of antimicrobials as appropriate to the target micro-organisms Antimicrobials are usually administered either orally (by gavage or in-feed or in-water) or parenterally by subcutaneous, intramuscular or intravenous routes. Ultrasound guided or laparoscopic direct injection into the developing egg in the hen is also possible.

Ideally, the egg is surgically removed in a sterile manner from the abdomen of the parent bird.

The invention further provides a method in which the sterile surgical removal comprises:—
performing a laparotomy incision and tying off the oviduct of the bird at both ends with sutures;
transecting the oviduct distal to each suture;
removing the egg enclosed in the oviduct;
sterilising the oviduct;
removing the egg; and
sterilising the egg.

Alternatively, the surgical sterile removal method may comprise:
making an incision in the skin of the bird;
manipulating the uterus to the surface; and
making an incision in the uterus and removing the egg and the uterus or clamping the uterus and removing the egg.

When the uterus is clamped and the egg is removed, the uterus may be repaired so that the bird may be able to lay more eggs. This aspect is important in the situation where the parent bird is valuable and should not be sacrificed. In this situation the bird is anaesthetised. Alternatively, the bird may be euthanized before sterile removal of the egg.

Sterile surgical removal of the egg is best completed rapidly, at about less than 30 minutes from time of euthanasia or anaesthesia, to avoid impairment of embryo viability. The prolonged use of anesthetics or excessive delays between euthanasia of the parent bird and removal of the egg will adversely affect embryo viability.

The egg once removed from the parent bird is then incubated in a sterile environment and hatched to produce a laying bird.

Essentially, what the present invention does is to provide the use of artificially derived eggs from parent birds in the production of eggs and derived birds to give laying birds for the control of micro-organisms. The said eggs and birds are as appropriate to their utility subsequently hatched, reared, maintained and bred, either conventionally, or in some form of isolator or sterile environment.

Ideally, one raises the bird as a parent bird in a sterile environment, feeding the bird with sterile food. Then, the egg is removed from the parent bird artificially prior to the transfer of the egg to an area of potential contamination in the parent bird and then the egg is incubated and hatched to produce a laying bird which is kept in this sterile environment.

In one embodiment of the invention the parent bird is chosen from a flock of similar birds all reared under the same conditions.

In another embodiment of the invention, the parent bird is hatched naturally in a sterile environment from a flock of birds of similar existing contamination free status.

In a further embodiment of the invention the parent bird is one of a flock of birds which are of another contaminant free status having been produced by suitable selection and natural rearing methods under controlled conditions and the method is used to provide birds of a different contaminant free status.

Preferably a laying bird forms part of a flock and after the laying birds are hatched, a sample of the laying birds is removed and tested for specific contaminants to provide a measure of the contaminant free status of the flock. Ideally when the specified contaminant free status is not achieved in the laying bird, the laying bird is used as a parent bird in the method. By an iterative process, it will be possible to eventually produce a flock of birds which will be virtually sterile and of germ-free status.

In one embodiment the laying bird is removed from the sterile environment to lay eggs which are, in turn, hatched to produce further laying birds.

In another embodiment the laying bird is removed from the sterile environment and fed with food containing non-pathogenic normal gutflora. The birds produced by this method, having normal gutflora, can be maintained at low cost and are suitable for consumption or use in the food industry.

In a further embodiment, the germ-free eggs of chickens or adult birds may be infected with selected non-pathogenic organisms (including potential probiotic bacteria and parasites), or with selected pathogens or with combinations of non-pathogens and pathogens. The eggs or birds so produced can be use, for example, for the scientific investigation of host-pathogen interactions, host-commensal interactions and the discovery and development of novel disease treatment and prevention methods and products for application in either birds or mammals.

Typically the bird is a chicken. Although this method may be carried out on all birds.

While in the above, the description has related entirely to poultry and specifically hens, it will be appreciated that the present invention may be carried out on other birds.

Preferably when a bird is hatched from a laying bird having the specified contaminant free status and is not a laying bird, the bird so laid is reared in a sterile environment for subsequent fertilisation of laying birds of the same or lower contaminant free status.

The invention also provides an egg and a bird produced by any of the methods of the invention.

According to a further aspect of the invention, the invention further provides a method of providing an egg of a specified contaminant free status comprising in a sterile environment:
housing a laying bird having the same or better contamination free status as provided in accordance with the method of the invention;
using the laying bird to lay the egg; and
removing the egg to another sterile environment.

Ideally the egg is, on laying, immediately removed and the shell of the egg is sterilised.

The laying bird may then be used to lay an egg, which may be the end product itself, or which may hatch into a bird which could either form a flock of birds of germ-free status or if it is not a laying bird, be used to fertilise a laying bird.

Preferably, the bird is anaesthetised or sacrificed by euthanasia or killing prior to removal of the egg in its shell. Female parent birds may be either live or recently killed. Live birds may, as consistent with ethical, legal and animal welfare considerations, be fully conscious, sedated or anaesthetised. Eggs and ova may be either fertilised or unfertilised.

Infectious organisms that may be controlled by the invention include organisms that can be pathogenic or non-pathogenic to the relevant species. These include avian species (typically chickens, fowls and turkeys), humans and other mammals (typically dogs, cats, horses, cattle, pigs, sheep, goats, rats and mice). For the purposes of the invention, micro-organisms include parasites, bacteria (including anaerobic and aerobic species, commensal species and species associated with the gut), mycoplasma, viruses (including retroviruses), prions, fungi, yeasts, moulds and DNA and RNA fragments.

If fertile eggs are used to produce offspring or derived birds, then the eggs may be hatched, reared, maintained and bred in either conventional husbandry systems, SPF systems or in isolators to control the entry of micro-organisms and maintain the germ-free status.

According to the invention, for maximum freedom from micro-organisms eggs should preferably be derived aseptically from parent females (unless they are also germ-free or gnotobiotic) and the life-cycle should be completed in isolators. The life-cycle may be completed outside isolators when SPF eggs and birds are produced.

According to the present invention, the aseptic derivation of eggs and, if appropriate hatching, rearing, maintenance and breeding of birds may be used in combination with another method of controlling microbial contamination. Such methods include disinfectants, antimicrobials, antibiotics, antiviral agents, antiparasitics, immunomodulators and vaccines.

It will be appreciated that in certain circumstances, when taking selected birds as parent birds, the laying birds produced may not in fact be sufficiently free of contaminants to produce laying birds of the right quality. It may then be necessary to carry out the same steps again using the eggs produced from such laying birds and artificially removing the eggs from these laying birds to provide further laying birds which hopefully will be contaminant free.

It will be appreciated that when birds are hatched which are not laying birds, they will then be retained for subsequent fertilisation of the laying birds. In this way, the whole flock can be sterile.

It will be possible, in the present invention, to produce simply the eggs for subsequent use. When eggs are required of a germ-free status, the first thing to do is to incubate the eggs derived surgically from the parent birds.

Rearing and breeding a bird in a healthy and productive state whilst maintained in a specified contamination free or sterile environment requires specialised diets to compensate for the lack of certain nutrients normally produced by, for example, the contaminants found in the gut or on the skin of a bird in a conventional environment.

According to a still further embodiment of the invention, there is provided a method for obtaining therapeutic and prophylactic biological products derived from sterile eggs produced in accordance with the invention. Such products include vaccines (live and killed), antibodies, monoclonal antibodies such at interferon, therapeutic and prophylactic proteins and other similar biological products which are all produced by well known techniques. Antigens for serological testing or other diagnostic tests may also be produced. The eggs may be used for tissue/cell culture and media production and in research use. The use of eggs for the production of, for example monoclonal antibodies, has the advantage that the antibodies or other proteins produced have high activity, are in the same form as human antibodies in terms of, for example, glycosylation. Furthermore costs relating to purification of the resultant protein product/monoclonal antibody may be reduced since the removal of live microrganisms, and toxic or contaminating products derived there-from requires additional, and often expensive, processing which also frequently causes lower yields and/or potency of the target protein.

Optionally, the parent bird may be a transgenic bird (i.e. a bird transgentically modified to carry exogenous DNA) which will give rise to an egg which produces an exogenous protein or other substances.

There is a risk of contaminating vaccines with pathogens that are transmitted through embryonated eggs. In the past, in order for vaccine production to be successful, using embryos from specific pathogen free (SPF) flocks minimized the risk and alternatively or additionally the flocks were kept in very clean environments. This new method of producing eggs of germ-free status ensures that the eggs used in vaccine production are sterile and overcomes contamination and sterility problems which can result in vaccines deemed unsafe for human or animal use. This new method essentially provides an alternative source of sterile eggs which are not contaminated by microorganisms. This has the advantage that it overcomes problems previously encountered with respect to contamination.

Eggs produced in accordance with the invention may be used to isolate a pathogenic micro-organism, produce a vaccine, antibodies, monoclonal antibody and other therapeutic and prophylactic molecules such as peptides and proteins and produce antigens for use in serological tests.

Egg production of a vaccine against a micro-organism such as a virus generally occurs via the following method. The appropriate virus strain is selected and then adapted to grow in eggs. These adapted virus strains are injected into fertilized eggs, which are subsequently incubated to enable growth of the virus. Large quantities of the virus can then be harvested from the egg before it reaches the hatching stage. The harvested virus can then be mixed and processed through several steps to eventually produce a fully formulated vaccine for either human or animal vaccination as appropriate. Large batches of up to several million eggs are harvested, processed and blended to form a vaccine product. Substantial numbers of eggs are used for the creation of virus seeds to initiate the infection of the eggs with the target virus and also to evaluate the safety of the vaccine, for example to confirm absence of adventitious agents or inappropriate processing of the target virus in the vaccine.

It will be understood that the vaccine may be composed of killed pathogenic micro-organisms, live strains of the micro-organism, recombinant strains, sub-units and/or isolated specific antigens.

According to a still further aspect of the invention there is provided a method for the production of microbiological semi-sterile birds or birds specified free of specific pathogens for replenishment of bird stocks. This is particularly valuable where conventional breeding and/or SPF flocks have broken down and become infected with specified pathogens. In these circumstances one of the most rapid and effective methods to re-create SPF flocks is to derive a new flock from existing infected flocks by using these to produce germ-free eggs derived in a sterile manner from screened and selected birds from the infected flock. This approach is particularly valuable when the infected birds are of special genetic or transgenetic merit. This is particularly applicable to bird stocks which have been infected with for example, avian flu virus or some bacterial species such as salmonella.

Avian influenza or “bird flu” is a contagious disease of fowl. While all bird species are thought to be susceptible, domestic poultry flocks are especially vulnerable to infections that can rapidly reach epidemic proportions. The most important control measures include rapid destruction of exposed birds, proper disposal of carcasses and the quarantining and rigorous disinfection of remaining bird stocks. The method of the present invention for the production of germ-free birds is applicable to the situation where there has been an outbreak of such a virus and the bird stocks need to be replenished. This method provides a safe and reliable method for re-establishing bird stocks which are sterile or at least semi-sterile or of specified status including freedom from specific micro-organisms including avian influenza virus.

The method for the production of germ-free, semi-sterile or specific pathogen free eggs for replenishment of bird stocks comprises the method of any of the preceding claims wherein the parent bird is obtained from either the flock of birds to be replenished or a suitable alternative, the laying bird produced therefrom and according to the method of the invention is tested to provide a measure of germ-free status and once the desired germ-free status has been obtained the laying bird is used to produce using the claimed method, progeny to form a flock of birds of appropriate germ-free status.

Alternatively, the bird and eggs may be provided for food and human consumer use, or animal use such as in a specified situation for particularly delicate patient with greater than usual susceptibility to infection or for any animal or human where avoidance of infection is desirable.

It will be understood that this invention applies to all avian and reptilian species, including but not limited to chickens, turkeys, quail, ducks, geese, guinea fowl, pheasant, partridge, parrots and grouse.

The invention will be more clearly understood from the following description of the method according to the present invention.

### Example 1

### Method

One flock of fifty adult female and five adult male chickens of known SPF status were maintained on selected diets and allowed to breed naturally. Timing of egg laying (oviposition) was recorded individually for each female over a two-week period. The mean time of day (time, L) when an egg was laid was calculated for each female. The time of day for L-3 h was calculated and the period from L-3 to L was nominated as the derivation interval. This interval was the time in which aseptic surgical laparotomy was performed for removal of the most developed eggs in each bird.

For the procedure, birds were euthanased by cervical dislocation and shortly afterwards prepared. Birds were submerged in a disinfectant solution for 5 minutes. Feathers were removed from the ventral thorax and abdomen and the exposed skin sterilised using a 50% solution of iodine in alcohol heated to 37° C. Each bird was then placed under a specially adapted surgical isolator sterilised with a 5% solution of peracetic acid and containing sterile instruments and a 500 ml flask containing iodine in alcohol. The bird was covered with a sterile drape and a sterile entry port of the isolator was then placed over the drape. A laparotomy incision was made and the oviduct (typically the uterus) was tied off at both sides of the egg using suture material. The oviduct was then transected distal to each of the sutures from the egg and the oviduct containing the egg was removed from the females' abdomen. The uterus-enclosed egg was then placed in the iodine/alcohol solution for five minutes after which the oviduct-enclosed egg was transferred via an entry port from the surgical isolator to a receiving isolator. In the receiving isolator, the oviduct was incised, the egg removed, swabbed with a disinfectant solution and transferred to an isolator adapted as a hatchery incubator.

Within one day of hatching, live chickens were removed from the hatchery isolator and transferred to two large-scale rearing isolators suitable for rearing groups of young chickens. Chickens were reared on commercial diets sterilised by radiation. At 18 days of age, five chickens were removed from each of the rearing isolators, euthanased and sampled for bacteriology by aerobic and anaerobic culture. Samples included liver, spleen, heart blood, vagina/cloaca, caecal and small intestinal digesta and faeces.

### Results

Viable chickens were hatched successfully from the artificially derived eggs (hatchability >50% more often >90%). No anaerobic or aerobic bacteria were isolated from the chickens sampled.

### Conclusion

A safe method for artificial production of germ-free fertile eggs in chickens was established. Eggs were viable and produced viable germ-free chickens which were successfully maintained in isolators. The method above, disclosed in European patent application no. 01650109, clearly enabled the production of germ-free chicks. However, repeated use of the method indicated that results were highly variable and often gave relatively low hatchability (e.g. <30%) which is not generally acceptable commercially.

### Example 2

A series of further studies, carried out over a period of 2 years, were conducted in accordance with the protocol of Example 1 in order to improve and refine the method of Example 1.

A total of 106 birds, in six flocks of varying ages and genotypes, were used in a series of small studies (each using 9 to 20 birds) to derive germ-free eggs. During this time results for both hatchability and microbiological germ-free status of chicks were very variable with hatchability ranging from zero to 40%. This was markedly lower than previously found. Another confounding factor was the failure in some studies to obtain germ-free status owing to microbiological contamination during the procedures to derive the egg. As a consequence of this the time taken to derive an egg after induction of either anaesthesia or euthanasia often increased in an effort to ensure sterility.

A series of investigations were therefore made (Example 2A-D) to evaluate the effects of various differences in egg removal procedure on hatchability and microbiological status of chicks from derived eggs. These variables included time from euthanasia or anaesthetic induction to removal of egg from the parents' abdomen, and timing of egg removal relative to anticipated time of egg laying and stage of development/maturity of the egg in the reproductive tract. Assessments included immaturity of egg (e.g. soft shells or egg too immature to remove intact), hatchability, sterility and ease of manipulation of the tissues (the latter causing a substantial increase in the time from euthanasia/anaesthesia to removal of an egg). Variables were evaluated individually.

Variables which affect sterility and viability of fertile eggs derived by sterile laparotomy were evaluated in this example. Evaluations made in the studies and the results obtained are provided below.

### Example 2A

Effects of difference in time between expected ovi-position and sterile surgical removal of the egg from the parent bird on egg viability and sterility and ease of surgical manipulation.

### Results

### Hatchability:

control (naturally laid eggs) 85-100% hatch, 80-100% sterile;
eggs removed from uterus within 30 min of anaesthesia or euthanasia, 13-40% hatch, 80-100% sterile;
eggs removed from uterus 60 min after euthanasia, 14% hatch, 80-92% sterile.

These results establish the viability of premature eggs in hens decreases with time after euthanasia.

### Ease of Surgical Manipulation:

Control not applicable;

30 minutes as above, good, tissues easily elevated;
60 minutes as above, difficult, tissues difficult to elevate /early rigor mortis.

These results establish that sterile surgical removal of eggs in a germ-free surgical isolator is facilitated by completion of surgery within 30 minutes.

### Example 2B

Effects of timed ovi-position versus palpation versus combination of these techniques on proportions of eggs with complete shells versus soft shells, viability (usually live chicks at time of hatching) and on ease of surgical manipulation.

### Results,

Timing alone, soft shells and no suitable egg for removal 8-71%, viability 13-50%, sterility 75-100%, ease of surgical manipulation, variable;
Palpation alone, soft shells & no suitable egg for removal 13-71%, viability 13-54%, sterility 89-100%, ease of surgical manipulation, good;
Timing combined with palpation, soft shells and no suitable egg for removal 10-23%, viability 14-57%, sterility 92-100%, ease of surgical manipulation, good.

These results suggest that palpation or palpation combined with timing may have some advantages over timing alone especially in terms of ease of manipulation when removing the egg from the abdomen. Further analysis of data indicated that timing plus palpation tended to be associated the shortest times for egg removal and that timing alone was associated with the longest times probably reflecting fewer soft shells in the timing plus palpation group. Fewer soft shells would facilitate easier and more rapid manipulation during egg removal.

### Example 2C

Effects of antibiotics (e.g. orally administered fluroquinolones) on elimination of transovarian bacterial and mycoplasmal infections and on viability and sterility of embryos and subsequent chicks.

### Results.

Without antibiotics, viability 22-60%, and sterility 66-92%;
With antibiotics, viability 13-57%, and sterility 89-100%.

These results establish the benefit of using antibiotics to remove transovaraian infection (e.g. Salmonella) and absence of adverse effects on viability of germ-free derived eggs.

### Example 2D

Effects of time (zero to 180 minutes) between euthanasia and egg removal on viability and ease of manipulation, hatchability and sterility

### Results.

### Sterility 100%,

Manipulation increasingly difficult after 30 minutes,
Hatchability was 20, 40 and 80% for eggs removed 60, 40 and 20 minutes after euthanasia (n=10 per time).

These results establish that the sterile removal of eggs from the abdomen of birds in a germ-free surgical isolator is facilitated by completion of surgery within approx 30 minutes.

### Example 3

A review of the results from the investigations in Examples 2A to 2D suggested that the greatest hatchability and microbiological sterility of derived eggs was most likely to be obtained with euthanized birds (anaesthetized birds being more difficult to manipulate), in which the egg was removed as quickly as possible after euthanasia (within 15-30 minutes) and in which the egg shell was firmly and well formed (maturation of egg well advanced, no soft shells) based on a combination of timed egg laying and manual palpation. Based on other data used to define optimal incubation conditions for premature derived eggs, a low (approx 25%) relative humidity was selected for the first 18 days of incubation. This method outlined below was a confirmatory investigation where all the variables identified in Example 2 were put together in an optimum manner to assess the interactions between these variables.

### Method and Materials:

A total of 180 adult female breeding chickens of known SPF status in 7 successive groups (of varying ages and genotypes) were maintained on selected diets and allowed to breed naturally. A combination of timing and palpation was used to determine the optimal time for egg removal from the birds abdomen.

For the procedure, birds were euthanized by cervical dislocation and immediately prepared for egg removal. Feathers were removed from the ventral thorax and abdomen and the exposed skin sterilised using a 50% solution of iodine in alcohol. Each bird was then placed under a specially adapted surgical isolator sterilised with a 5% solution of peracetic acid and containing sterile instruments. The bird was covered with a sterile adhesive drape and a sterile entry port of the isolator was then placed over the drape. A laparotomy incision was made and following careful dissection the egg was removed from the uterus/distal aspect of the reproductive tract and proximal to the cloaca. The egg was then transferred under germ-free conditions to an isolator adapted as a hatchery incubator. A total of 153 eggs with good shells were successfully recovered, 13-22 minutes after euthanasia, and were considered suitable for incubation.

After storage for approximately 6-24 h, at 18-20C in a vibration-free, ventilated plastic egg tray, the eggs were weighed and then the eggs were placed in small rocking incubators. The incubators were maintained at a RH of approx 25% RH and 37.6C. Eggs were held air cell uppermost and were weighted individually on days 0, day 7 and 18 of the incubation. On day 18, all incubators where adjusted to provide a RH of approx 65%. Numbers of eggs that pipped and of live viable chicks were recorded.

Within seven days of hatching, live chickens were removed from the hatchery isolator and transferred to larger-scale rearing isolators suitable for rearing groups of young chickens. Chickens were reared on supplemented commercial diets sterilized by radiation. When the chicks were 14-21 days of age, fecal samples and swabs were removed from each of the rearing isolators and sampled for bacteriology by aerobic and anaerobic enriched culture.

### Results:

Viable chickens were hatched successfully from the artificially derived premature eggs. No anaerobic or aerobic bacteria were isolated from the samples. (Eggs laid naturally by the parent birds within 9 days prior to euthanasia had a viable chick hatchability of 86%. This confirms a normal or high level of natural fertility in the eggs produced by the females)

The hatchability of the 7 groups of germ-free eggs ranged from 60% (9 of 15) to 77% (17 of 22), with a mean of 74%.

### Conclusion

A safe and highly effective method for artificial production of germ-free fertile eggs in chickens was established. Eggs were fertile and produced viable germ-free chickens which were successfully maintained in isolators. This study confirmed using a large number of eggs across 7 successive flocks of chickens that, compared with methods described previously for the production of germ-free fertile eggs, less variability in hatchability of derived eggs and 100% consistent germ-free microbiological status can be obtained by selecting birds with an egg that is well matured (close to time of natural laying; selection based on combination of timing of egg laying and palpation), rapidly removing the egg from the parent female bird (15-30 minutes from start of euthanasia or anaesthesia) and then incubated at RH of approx 25% (much lower than for naturally laid eggs).

Using the techniques disclosed in the present application, we are able to achieve much more consistent high hatchability rates, that is hatchability rates consistently greater than 60%. This was not possible before on such a consistent basis.

1. A method for increasing the hatchability and viability of eggs of germ-free status when obtained from a parent bird which is carried out in a sterile environment prior to removal of a premature egg in its shell from a parent bird and which includes the following steps:
(a) housing a bird as a parent bird;
(b) determining the timing of when to remove the premature egg from the parent bird by:
(i) determining the position of the premature egg in the reproductive tract of the parent bird prior to transfer of the premature egg to the cloaca of the parent bird; and
(ii) establishing when the requisite level of egg-shell formation and calcification has taken place by determining the stage of development of the premature egg in terms of shell formation and calcification.

2. (canceled)

3. (canceled)

4. (canceled)

5. (canceled)

6. (canceled)

7. A method of rearing a bird of germ-free status comprising, in a sterile environment, comprising the steps:
a) housing a bird as a parent bird;
b) determining the timing of when to remove the premature egg in its shell from the parent bird by:
i. determining the position of the premature egg in the reproductive tract of the parent bird prior to transfer of the premature egg to the cloaca of the parent bird; and
ii. establishing when the requisite level of egg-shell formation and calcification has taken place by determining the stage of development of the premature egg in terms of shell formation and calcification;
c) removing the premature egg in its shell from the parent bird when the requisite level of egg-shell formation and calcification has taken place and prior to transfer of the premature egg to the cloaca in the parent bird; and
d) incubating the premature egg in its shell and hatching the premature egg to produce a laying bird.

8. The method according to claim 7 wherein step (b) occurs by a combination of observation and/or physical methods, wherein said physical methods include abdominal and pelvic palpation, ultrasound, X-ray and/or MRI scanning.

9. (canceled)

10. The method according to claim 7 wherein the method enables, the determination of the location of the premature egg prior to the pelvic outlet of the parent bird prior to removal of the premature egg from the parent bird, and wherein the premature egg is removed when the premature egg is substantially half way between the caudal aspect of the sternum and the tuber ischii of the parent bird.

11. (canceled)

12. The method according to claim 7 wherein the observation methods include the steps of monitoring and recording the laying pattern of the parent bird over time to produce an estimated transfer time of the egg to the distal reproductive tract and cloaca.

13. The method according to claim 7 in which the removal of the premature egg is at a time prior and as close as possible to the transfer time when the egg would transfer naturally to the cloaca in the parent bird.

14. The method according to claim 7 further comprising the step of removing micro-organisms which infect the premature egg whilst in the reproductive tract, comprising the steps:
i. identification of target micro-organisms which infect the premature egg whilst in the reproductive tract;
ii. selection of appropriate antimicrobials to combat the target micro-organisms, such as fluroquinolone, cephalosporin and macrolide antimicrobials and
iii. administration of the antimicrobial to the parent bird and/or to the premature egg in-ova.

15. (canceled)

16. (canceled)

17. (canceled)

18. The method according to claim 7 wherein the parent bird is sacrificed, euthanased or anaesthetised and removal of the premature egg in step (c) is carried out less than approximately 30 minutes from the time of sacrifice, euthanasia or anaesthesia of the parent bird.

19. The method as claimed in claim 7 in which the parent bird is chosen from a flock of similar birds all reared under the same conditions

20. The method as claimed in claim 7 in which the parent bird is hatched naturally in a sterile environment from a flock of birds of similar existing contamination free status.

21. The method as claimed in claim 7, in which the parent bird is one of a flock of birds which are of another contaminant free status having been produced by suitable selection and natural rearing methods under controlled conditions and the method is used to provide birds of a different contaminant free status.

22. The method as claimed in claim 7, in which laying bird forms part of a flock and after the laying birds are hatched, a sample of the laying birds is removed and tested for specific contaminants to provide a measure of the contaminant free status of the flock.

23. The method as claimed claim 7, in which when the specified contaminant free status is not achieved in the laying bird, the laying bird is used as a parent bird in the method.

24. The method as claimed claims 7 in which the egg is surgically removed from the abdomen of the parent bird, comprising the steps:
i. making an incision in the skin of the bird;
ii. manipulating the uterus to the surface; and
iii. making an incision in the uterus and removing the egg or clamping the uterus and removing the egg.

25. (canceled)

26. The method according to claim 7 wherein the removal of the premature egg from an anesthetized, euthanized or sacrificed parent bird occurs in a sterile environment and the sterility of the procedure is checked throughout the procedure.

27. The method as claimed in claim 7 in which the laying bird is removed from the sterile environment to lay eggs which are, in turn hatched to produce further laying birds.

28. The method as claimed in claim 7 in which the laying bird is removed from the sterile environment and fed with food containing normal gutflora.

29. (canceled)

30. The method as claimed in claim 7, in which when a bird is hatched from a laying bird having the specified contaminant free status and is not a laying bird, the bird so laid is reared in a sterile environment for subsequent fertilisation of laying birds of the same or lower contaminant free status.

31. A method of providing an egg of germ-free status in a sterile environment comprising
i. housing a laying bird having the same or better germ-free status as provided in accordance with the method of claim 7;
ii. using the laying bird to lay the egg; and
iii. removing the egg to another sterile environment

32. The method as claimed in claim 31 in which the egg is, on laying, immediately removed and the shell of the egg is sterilised.

33. An egg produced in accordance with the method of claim 31 or 32.

34. A bird hatched from an egg as claimed in claim 33.

35. A bird hatched in accordance with the method of claims 7.

36. A bird hatched from an egg laid by a laying bird reared in accordance with the method of claim 7.

37. Use of eggs produced in accordance with claim 7 in the production of therapeutic and prophylactic biological substances, such as vaccines, antibodies, monoclonal antibodies, fibroblasts, proteins and/or antigens for serological testing or other similar protein products.

38. Use according to claim 37 in the production of a vaccine wherein the egg is injected with a selected virus strain, the egg is then incubated to produce a virus and the virus produced therefrom forms a vaccine.

39. Use according to claim 37 wherein the egg produces an exogenous protein.

40. Biological products for use in therapeutic and prophylactic methods, prepared using an egg as produced by the method of claim 31, such as a vaccine antibody monoclonal antibody fibroblast, protein and/or antigen for serological testing or other similar protein product preferably a vaccine for an influenza virus.

41. (canceled)

42. (canceled)

43. A method for the production of semi-sterile eggs for replenishment of bird stocks according to the method of claim 7 wherein the parent bird is obtained from the flock of birds to be replenished, the laying bird produced therefrom is tested to provide a measure of germ-free status and once the desired germ-free status has been obtained the laying bird and its progeny form a flock of birds of appropriate germ-free status.
